(19) United States
(12) Patent Application Publication
(10) Pub. No.: US 2010/0028449 A1
(43) Pub. Date: Feb. 4, 2010

(54) FERMENTED MILK PRODUCT AND USE THEREOF

(76) Inventors: Satya Prakash, Brossard (CA); Aleksandra Malgorzata Urbanska, Montreal (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/303,758

(22) PCT Filed: Jun. 6, 2007
(86) PCT No.: PCT/CA2007/001010

§ 371 Date: Apr. 1, 2009

Related U.S. Application Data
(60) Provisional application No. 60/804,008, filed on Jun. 6, 2006.

(51) Int. Cl. A61K 9/14; A61K 35/74; A61P 29/00; A61P 13/00; A61P 35/04; A61P 1/00

(52) U.S. Cl. 424/490; 424/93.4; 424/93.45

(57) ABSTRACT

The present invention relates to an oral formulation comprising a microcapsule containing bacteria and a fermented milk carrier. There is also provided a method of medical treatment of an inflammatory gastrointestinal disease or disorder in a subject in need thereof, comprising detecting the presence of inflammatory gastrointestinal disease or disorder in the subject, wherein if inflammatory gastrointestinal disease or disorder is detected, then administering the formulation of the present invention to the subject.

### BACKGROUND OF THE INVENTION

(a) Field of the Invention

The present invention relates to a novel method for encapsulating live bacteria; an encapsulated live bacteria; an oral formulation for probiotic therapy and method of treatment thereof.

(b) Description of Prior Art

A well balanced gut microflora is known to contribute to the maintenance of a healthy intestinal mucosa. The density of gastrointestinal (GI) microflora increases from the stomach to the large intestine reaching 1010-1012 cfu/g in the colon. One of the most important groups of bacteria for intestinal health is lactic acid bacteria (LAB) (Adolfsson, O. et al., (2004), American Journal of Clinical Nutrition 80:245-256). LAB are considered probiotic; live microorganisms that remain in the GI tract to benefit the host (Adolfsson, O. et al., (2004), American Journal of Clinical Nutrition 80:245-256; Roberfroid, 2000). Although their mechanism of action is not known, it is believed that LAB, like other probiotic microorganisms, compete and suppress the growth of undesirable microorganisms in the colon and intestines leading to the stabilization of the digestive system (Adhikari, K. et al., (2000), Journal of Dairy Science 83:1946-1951).

There are several reports that probiotic yogurt has significant clinical benefits (Donaldson, M. S., (2004), Nutrition Journal 3:19). It is estimated that a decrease of at least 60-70 percent in breast, colorectal, and prostate cancers and 40-50 percent in lung cancer would occur when a diet is complied with (according to the anti-cancer diet guidelines) which includes probiotic yogurt products. In order to be labeled probiotic, yogurt must contain a cell load of at least 107 cfu/g at the time of manufacture (Chandan, R. C. et al., (1993), Ed Hui, Y H, VCH Publishers, Inc, New York 1-56). However, it has been found that this level of live bacterial cells in probiotic yogurt is not adequate to provide the maximum benefit, especially considering that many bacteria do not survive storage (Donaldson, M. S., (2004), Nutrition Journal 3:19), (Dave, R. I. et al., (1998), Journal of Dairy Science 81:2804-2816), (Shah, N. P., et al., (1995), International Dairy Journal 5:515-521) or passage through the stomach.

Therefore several attempts have been made to deliver a greater number of live bacterial cells. One strategy to deliver more live bacteria to the intestines is bioencapsulation. This technology has developed over the last 20 years, (Orive, G. et al., (2003b), International Journal of Pharmaceutics 259:57-68; Prakash, S. et al., (1996b), Nature Medicine 2:883-887; Sun et al., 1987, Chang, T. M. S. et al., (1998), Molecular Medicine Today 4:221-227; Prakash, S. et al., (1998), Artificial Cells Blood Substitutes and Immobilization Biotechnology 26:35-51; Jones, M. L. et al, (2004), Journal of Biomedicine and Biotechnology 1:61-69). However, the use of this technology in probiotic yogurt formulation containing live bacteria has not yet been investigated.

The transit of free bacteria through the gastrointestinal tract is often problematic because of low pH conditions, enzymatic digestion and very few probiotic cells finally reach their targeted site. The challenge here consist in producing a support allowing successful storage and transport of bacteria which could, if added to one's diet, constitute an alternative but effective treatment to various medical issues caused by an imbalance between desirable and undesirable microorganisms in the GI microflora.

Therefore, it would be highly desirable to be provided with a novel method to encapsulate live bacteria and which would be safe for oral administration as a probiotic formulation to improve gastrointestinal microflora's condition.

### SUMMARY OF THE INVENTION

Herein we report the potential of microcapsules as a platform for probiotic live bacterial cell oral delivery. In vitro data suggests that capsules containing live Lactobacillus acidophilus cells showed superior mechanical stability and demonstrated significantly higher bacterial cell survivals compared to free bacterial cells over a period of 4 weeks. Using an in vitro simulation human stomach model, we monitored the survival rates of free and alginate-poly-L-lysine (PLL)-alginate (APA) membrane microencapsulated L. acidophilus cells at 37° C. over two hours, the approximate time it takes food to pass through the stomach. Results show that 7.10 log cfu/g of microencapsulated L. acidophilus cells were found alive compared to only 5.51 log cfu/g of free L. acidophilus cells in the presence of simulated gastric fluid (SGF) and 2% milk fat M.F. yogurt. In addition, data shows that only 6.66 log cfu/g of microencapsulated L. acidophilus cells survived in SGF fluid in the absence of yogurt. The high survival rates of encapsulated L. acidophilus cells strongly suggest the use of microcapsules and yogurt for probiotic bacterial cell delivery.

In accordance with the present invention there is provided an oral formulation to improve a patient gastrointestinal microflora, which comprises coated microcapsule containing bacteria in suspension in a probiotic acceptable carrier, wherein said coated microcapsule comprises an encapsulated bacteria in a semipermeable capsule coated with poly-L-Lysine (PLL) and alginate and is also resistant in gastrointestinal conditions.

The bacteria may be chosen from Lactobacilli cells, Bifidobacterium cells, Lactobacillus plantarum 80, Lactobacllus delbrueckii subsp. Lactis, Lactobacillus Rhamnosus, Lactobacillus, more particularly from Lactobacillus acidophilus, Lactobacillus casei, Bifidobacterium, Lactobacillus plantarum 80, Lactobacillus delbrueckii subsp. Lactis, Lactobacillus Rhamnosus, Lactobacillus GG.

In accordance with a preferred embodiment of the oral formulation of the present invention, the bacteria is live.

In accordance with another embodiment of the oral formulation of the present invention, the microcapsule is made of a material chosen from alginate-poly-L-Lysine-alginate (APA), alginate-chitosan (AC), alginate pectinate polylysine pectinate alginate (APPPA), alginate polyethylene glycol alginate (APEGA), alginate chitosan genipin alginate (ACGA).

In accordance with another embodiment of the oral formulation of the present invention, the probiotic acceptable carrier is at a substantially basic pH to further protect from gastrointestinal fluids.

In accordance with another embodiment of the oral formulation of the present invention, the probiotic acceptable carrier is chosen from a food supplement or food.

In accordance with another embodiment of the oral formulation of the present invention, the food carrier is chosen from yogurt, ice cream, cheese, chocolate, nutritional bars, cereal, milk, infant formulation, fruit juices.

In accordance with the present invention there is provided a method for probiotic therapy of a patient for improving gastrointestinal microflora, which comprises orally administering the oral formulation of the present invention.

In accordance with another embodiment of the method of the present invention, the patient is suffering from a disease or disorder chosen from breast cancer, colorectal cancer, prostate cancer, lung cancer, urinary tract infections, yeast infections and inflammatory bowel diseases (IBD), Crone's diseases (CD).

In accordance with another embodiment of the present invention, there is provided an oral formulation comprising:

a microcapsule containing bacteria; and

a fermented milk carrier.

The microcapsule may comprise a semipermeable capsule comprising poly-L-Lysine (PLL) and alginate and wherein the microcapsule is resistant to degradation in gastrointestinal conditions.

The bacteria may be Lactobacilli bacteria or Bifidobacterium bacteria. The Lactobacilli bacteria are selected from the group consisting of Lactobacillus acidophilus, Lactobacillus casei; Lactobacillus plantarum 80, Lactobacillus delbrueckii subsp. Lactis, Lactobacillus Rhamnosus,

In accordance with another embodiment of the present invention, there is provided a method for treatment or prevention of a disease or disorder in a subject in need thereof or for nutritional supplementation of a subject, comprising orally administering to the subject the oral formulation of the present invention.

In accordance with another embodiment of the present invention, there is provided the use of the oral formulation of the present invention for the preparation of a medicament for the treatment or prevention of a disease or disorder or for the preparation of a nutritional supplement.

In accordance with another embodiment of the present invention, there is provided a fermented milk carrier i) for use as a prebiotic carrier in increasing the efficacy of microencapsulated bacteria in the treatment of a disease or disorder in a subject or ii) for preparation of a medicament for the treatment of a disease or disorder in a subject; wherein optionally the carrier is used in the oral formulation of the present invention.

The subject may be a mammal, optionally a human.

The disease or disorder includes a gastrointestinal disease or disorder.

The gastrointestinal disease or disorder includes an inflammation gastrointestinal disease or disorder such as Inflammatory Bowel Disease (IBD), Crohn's Disease, colitis, enteroinvasive colitis, C. difficile colitis, Ulcerative Colitis (UC), Inflammatory Bowel Syndrome (IBS), pouchitis, diverticulitis, gastroenteritis, colic, appendicitis, appendicitis, ascending colangitis, esophagitis, gastritis, or enteritis.

The disease or disorder includes cancer, such as breast cancer, colorectal cancer, prostate cancer, lung cancer, colon cancer and inflammation-related colon cancer, including adenoma, carcinoma, leiomyosarcoma, carcinoid tumor, or squamous cell carcinoma. Lactobacillus GG, Bifidobacterium infantis, Bifidobacterium breve, Bifidobacterium longum, Bifidobacterium bifidum.

The bacteria may be live.

The bacteria may be present in a range from 109 to 1012 colony forming units (CFU).

The microcapsule may comprise a material selected from the group consisting of alginate-poly-L-Lysine-alginate (APA), alginate-chitosan (AC), alginate pectinate polylysine pectinate alginate (APPPA), alginate polyethylene glycol alginate (APEGA) and alginate chitosan genipin alginate (ACGA).

The fermented milk carrier may comprise a basic pH buffer and protects the bacteria and/or the microcapsule from gastrointestinal fluids. The basic pH buffer may be between pH 7-9.

The fermented milk carrier may comprise a food supplement or food, such as yogurt, cheese, milk, powdered milk, kefer or a fermented milk formulation.

The yogurt may be selected from the group consisting of plain yogurt, flavored yogurt, yogurt beverage, Dahi, Dadiah, Labneh, Bulgarian Yogurt, Tarator, Cacik, Lassi and Kefir.

The yogurt may comprise 1-10 grams of microencapsulated bacteria per 100 grams of yogurt, optionally 5-10 grams of microencapsulated bacteria per 100 grams of yogurt, optionally 8-10 grams of microencapsulated bacteria per 100 grams of yogurt.

The yogurt may comprise 4.2 grams of harvested bacteria in 100 mL of 1.65% alginate solution.

The oral formulation of the present invention may be use in nutritional supplementation of a subject or for use in preventing or treating a disease or disorder in a subject.

The disease or disorder includes inflammation of tissue in bowel, colon, sigmoid colon, rectum, appendix, anus, esophagus, stomach, mouth, liver, billiary, tract or pancreas, including inflammation of colon.

The inflamed tissue or colon comprises increased interleukins and cytokines compared to non-inflamed tissue or colon, such as up-regulated inflammatory response and markers compared to non-inflamed tissue or colon, such as tumor necrosis factor-α[TNF-α], interleukin-1 [IL-1], IL-6, IL-12, and γ-interferon in macrophages.

The disease or disorder includes a urinary tract related disease or disorder.

The urinary tract related disease or disorder includes a urinary tract infection or a yeast infection.

In accordance with another embodiment of the present invention, there is provided a method of medical treatment of an inflammatory gastrointestinal disease or disorder in a subject in need thereof, comprising detecting the presence of inflammatory gastrointestinal disease or disorder in the subject, wherein if inflammatory gastrointestinal disease or disorder is detected, then administering the formulation of any one of claims 1 to 13 to the subject.

The detecting step may comprise determining the presence of inflammatory gastrointestinal disease or disorder in the subject with a biopsy of the subject's tissue or a blood test of the subject, such as detection of: elevated C Reactive Protein (CRP), increased Erythrocyte Sedimentation Rate (ESR), elevated neutrophil count, elevated eosinophil count, elevated monocyte count, elevated white blood cell count (WBC), elevated immunoglobulin count or elevated IgA, compared to a subject not having inflammation.

In accordance with another embodiment of the present invention, there is provided a method of medical treatment of inflammation-related colon cancer in a subject in need thereof, comprising detecting the presence of inflammation-related colon cancer in the subject, wherein if cancer is detected, next administering the formulation of the present invention.

The detecting step may comprise determining the presence of cancer in the subject using fecal occult blood (FOB), visible protrusion adenomatous polyps from the mucosal surface, digital rectal exam, colonoscopy, sigmoidiscopy, abdominal series radiograph with contrast, double contrast enema abdominal radiograph or abdominal CT scan.

The detecting step may comprise determining the presence of cancer in the subject with a blood test of the subject comprising detection of elevated carcinoembryonic antigen (CEA) compared to a subject not having cancer.

The detecting step may comprise determining the presence of cancer in the subject with a biopsy of the subject's tissue or a blood test of the subject, such as detection of: elevated C Reactive Protein (CRP), increased Erythrocyte Sedimentation Rate (ESR), elevated neutrophil count, elevated eosinophil count, elevated monocyte count, elevated white blood cell count (WBC), elevated immunoglobulin count or elevated IgA, compared to a subject not having cancer, such as adenoma or carcinoma.

In accordance with another embodiment of the present invention, there is provided an oral formulation for the treatment and/or prevention of a disease and/or disorder, which comprises coated microcapsule containing bacteria in suspension in a fermented milk probiotic acceptable carrier, wherein said coated microcapsule comprises encapsulated bacteria in a semipermeable capsule coated with poly-L-Lysine (PLL) and alginate and is also resistant in gastrointestinal conditions.

For the purpose of the present invention the following terms are defined below.

The expression “mechanically resistant” is referring to an intrinsic construction's capacity of a microcapsule which allows to maintain its original structure and shape against physical and/or mechanical stresses in a particular environment.

The expression “gastrointestinal conditions” is referring to the various mechanical stresses of the gastrointestinal tract and to the different acidity levels of the gastrointestinal fluids which ingested substances undergo.

### BRIEF DESCRIPTION OF THE DRAWINGS

FIG. 1, left, illustrates freshly prepared empty APA microcapsules whereas FIG. 1, right, illustrates freshly prepared APA microcapsules loaded with L. acidophilus cells.

FIG. 2 is a photomicrograph of four different stages of APA microcapsules. In photomicrograph (a), freshly prepared empty APA microcapsules are shown whereas in photomicrograph (b), they were loaded with L. acidophilus. Photomicrograph (c) is an illustration APA microcapsules loaded with L. acidophilus cells after 76 hours of incubation in MRS broth and 370 rpm in vitro shaking at 37° C.

FIG. 3 illustrates empty APA microcapsules exposed to shaking at 150 rpm at 37° C. in three different conditions. In (a), the microcapsules were introduced in SGF (pH 1.98) for 3 hrs. In b), they were incorporated in SGF (pH 1.98) for 12 hrs and in (c), they were incorporated in SGF (pH 1.98) for 3 hrs and in SIF (pH 6.5) for 24 hrs.

FIG. 4 is a graph of the mechanical stability of empty APA microcapsules at various exposure times in simulated gastric fluid (SGF) (pH 1.98) and simulated intestinal fluid (SIF) (pH 6.5) after shaking at 150 rpm at 37° C.

FIG. 5 is a photomicrograph of various APA microcapsules loaded with L. acidophilus cells, exposed to mechanical shaking of 100 rpm at 4° C. and stored in various conditions. In Y1) storage was in 2% M.F. yogurt for 1 week. In P1), storage was in 0.85% physiological solution for 1 week. In Y2) storage was in 2% M.F. yogurt for 2 weeks. In P2) storage was in 0.85% physiological solution for 2 weeks. In Y3) storage was in 2% M.F. yogurt for 3 weeks. In P3), storage was in 0.85% physiological solution for 3 weeks. In Y4) storage was in 2% M.F. yogurt for 4 weeks and in P4), storage was in 0.85% physiological solution for 4 weeks.

FIG. 6 A) is a graph of the viability of live L. acidophilus cells in 2% M.F. yogurt during 4 weeks of mechanical shaking at 100 rpm at 4° C. FIG. 6 B) is a graph illustrating the retention capacity of APA microcapsules. The number of viable L. acidophilus bacteria in the supernatant of storage media gives an indication of how many L. acidophilus bacteria have leaked from the microcapsules. The APA microcapsules loaded with L. acidophilus cells were stored in 0.85% physiological solution for 4 weeks at 4° C. No mechanical stress was applied.

FIG. 7 is a graph evaluating the survival of APA encapsulated L. acidophilus cells in pH 2, 3, 4, 6 and 8 in presence of 2% M. F. yogurt at 37° C.

FIG. 8 is a graph effectuating a comparison of the survival of APA encapsulated and free L. acidophilus cells in conditions simulating the stomach supplemented with 2% M.F. yogurt at 37° C.

FIG. 9 displays photomicrographs of freshly encapsulated empty capsules and capsules loaded with L. acidophilus cells of 550±26 μm in size and magnification of 2.5× using light microscopy. Left: Photomicrograph of freshly prepared empty AC microcapsules (size 550±96 μm, magnification: 2.5×). Right: Photomicrograph of freshly prepared AC microcapsules loaded with L. acidophilus cells.

FIG. 10 shows three comparative photomicrographs of freshly prepared microcapsules. FIG. 10 (a) Photomicrograph of freshly prepared empty AC microcapsules. (b) Photomicrograph of freshly prepared AC microcapsules loaded with L. acidophilus. (c) Photomicrograph of AC microcapsules loaded with L. acidophilus cells after 76 hours of incubation in MRS broth and 150 rpm in-vitro shaking at 37° C. (Magnification: 2.5×).

FIG. 11 displays three photomicrographs of AC microcapsules exposed to simulated gastrointestinal fluid (SGF) (pH 1.98) for 3 hours (11a), to SGF for 12 hours (11b) and to simulated intestinal fluid (SIF) (pH6.5) for 24 hours. FIG. 11. Photomicrographs of AC microcapsules loaded with L. acidophilus cells exposed to shaking at 150 rpm at 37° C.: (a) in SGF (pH 1.98) for 3 hrs. (b) in SGF (pH 1.98) for 12 hrs. (c) in SGF (pH 1.98) for 3 hrs and in SIF (pH 6.5) for 24 hrs. (Magnification: 6.3×).

FIG. 12 further demonstrates physical property of exposed microcapsules to a combination of simulated fluids. Mechanical stability of AC microcapsules loaded with L. acidophilus cells at various exposure times in simulated gastric fluid (SGF) (pH 1.98) and simulated intestinal fluid (SIF) (pH 6.5) after shaking at 150 rpm at 37° C.

FIG. 13 illustrates the survival of encapsulated bacterial cells in SGF with and without addition of 2% M.F. yogurt as well as the survival of free bacteria contained in the yogurt. Comparison of the survival of AC (chitosan 10) encapsulated and free L. acidophilus cells using Simulated Human Intestinal Microbial Ecosystem—conditions simulating the stomach supplemented with 2% M.F. yogurt at 37° C.

FIG. 14 displays survival of AC encapsulated and free bacterial cells obtained by exposure to simulated intestinal fluid conditions. Comparison of the survival of AC (chitosan 10) encapsulated and free L. acidophilus cells using Simulated Human Intestinal Microbial Ecosystem—condition simulating the intestines supplemented with 2% M.F. yogurt at 37° C.

FIG. 15 is a comparative study—survival of AC 10 encapsulated L. acidophilus in presence and of 2% M.F. yogurt at 4° C. and mechanical shaking of 100 rpm.

FIG. 16 illustrates comparative study of microencapsulated L. acidophilus bacterial cells viability in various chitosan concentrations and polymers (0.5%/10, 0.25%/10, 0.1%/10) in 2% M.F. yogurt with free L. acidophilus bacterial cells in 0.85% saline during 4 weeks of mechanical shaking at 100 rpm at 4° C.

FIG. 17 Viability of free L. acidophilus cells in 2% M.F. plain yogurt in buffers: pH2, pH3, pH4, pH6 and pH8.

FIG. 18 Survival of AC encapsulated live L. acidophilus cells in buffers: pH2, pH3, pH4, pH6 and pHB supplemented with 2% M.F. plain yogurt.

FIG. 19 is a photomicrograph of APA microcapsules loaded with Lactobacillus acidophilus bacterial cells at 77× magnification and (b) at 112× magnification. (size 433 um±67)

FIG. 20 illustrates the effect of the treatment on animal body weights in the Min mouse. Data represent the mean±SEM per group.

FIG. 21 illustrates the changes in the expression levels of anti-inflammatory interleukin-6 examined during treatment at different time intervals. The data represent the mean±SEM of expression levels per group.

FIG. 22 illustrates the effect of the treatment on total fecal bile acid levels. The data represent the mean±SEM of expression levels per group.

FIG. 23 illustrates the number of adenoma (a) and Gastrointestinal Intraepithelial Neoplasias (b) for three groups: Control—gavaged empty APA microcapsules+0.85% saline, Treatment 1—gavaged L. acidophilus bacterial cells in APA microcapsules+2% M.F. yogurt and Treatment 2—gavaged L. acidophilus bacterial cells in APA microcapsules+0.85% saline found in the large intestines. Data represent the mean±SEM per group.

FIG. 24 illustrates the number of adenoma (a) and Gastrointestinal Intraepithelial Neoplasias (b) for three groups: Control—gavaged empty APA microcapsules+0.85% saline, Treatment 1—gavaged L. acidophilus bacterial cells in APA microcapsules+2% M.F. yogurt and Treatment 2—gavaged L. acidophilus bacterial cells in APA microcapsules+0.85% saline found in the small intestines. Data represent the mean±SEM per group.

FIG. 25 illustrates histological sections showing intestinal changes in C57BL/6J-Apc^{Min/+} mice. FIG. 25 (a) consists of a representative tumor of the colon found in a control untreated mouse shows pedunculated (polypoid) adenoma with high grade of dysplasia. Original magnification 40×. FIG. 25 (b) consists of gastrointestinal intraepithelial neoplasia (microadenoma) of the small intestine found in a mouse gavaged with L. acidophilus bacterial cells in APA microcapsules+2% M.F. yogurt. Note the increased Nuclear/Cytoplasmic ratio, the nuclear crowding and the hyperchromasia of these glands (arrow). Original magnification 100×. FIG. 25 (c) consists of papillary Adenoma in small intestine, sessile with low grade of dysplasia (arrows) (Sessile adenomatous polyp) found in a mouse gavaged with L. acidophilus bacterial cells in APA microcapsules+2% M.F. yogurt 0.85% saline. Original magnification 100×. FIG. 25 (d) consists of broad-based adenoma of small intestine found in a mouse gavaged with L. acidophilus bacterial cells in APA microcapsules+0.85% saline. Original magnification 100×. All tissues were stained with hematoxylin eosin.

### DETAILED DESCRIPTION OF THE INVENTION

In accordance with one embodiment of the present invention, the bacteria to be encapsulated is chosen from any Lactobacilli and any Bifidobacterium. Known such bacteria include L. casei, L. acidophilus, L. plantarum, L. fermentum, L. brevis, L. jensenii, L. crispatus, L. rhamnosus, B. Iongum and B. breve. The preferred bacteria used in accordance with the present invention are L. acidophilus, L. casei and Bifidobacterium bifidus.

In accordance with another embodiment of the present invention, the microencapsulated bacteria are coated with a 0.1% PLL and 0.1% alginate solution. Accordingly, the present invention is effective with any microcapsules.

In accordance with another embodiment of the present invention, the encapsulated live bacteria may be suspended in a probiotic acceptable carrier. Such carrier is chosen, without limitation, from a food supplement or food. More preferable, it can be chosen from yogurt, ice cream, cheese, chocolate, nutritional bars, cereal, milk, infant formulation, fruit juices. In cases of dairy products specially youghurt, its composition such as nutrients (ylamines, metal ions, cofactors, proteins, fat contents, sugars, etc) will provide a further protection for the encapsulated live bacterial cells from the gastric fluids and other gastrointestinal environments.

### Materials and Method

Sodium alginate (low viscosity), poly-L-lysine (MW=27,400) (lot 71K5120) and calcium chloride (desiccant, 96+%, A.C.S. reagent, FW 110.99, d 2.15, batch # 05614AC) were purchased from Sigma-Aldrich, Canada. MRS AGAR Difco™ Lactobacilli and MRS BROTH Difco™ Lactobacilli were purchased from Becton, Dickinson and Company Sparks, USA. Liberty plain yogurt 2% M.F. containing active Acidophilus and Bifidus cultures was procured from a local store.

Bacteria Cultures, Propagation and Enumeration.

L. acidophilus (ATCC 314) cells were inoculated in 100 mL of MRS broth. The bacteria were then cultured in MRS Broth at 37° C. in a Professional Sanyo MCO-18M Multi-Gas Incubator. Cultures were grown for 24 hours and centrifuged at 3000×g for 15 minutes at 37° C. The media was decanted; the cells were suspended in 100 mL of fresh MRS media and incubated for an additional 20 hours at 37° C. After growth was performed, the resulting cell wet weights were noted. Anaerobic jars and gas generating kits (Atmosphere Generation System AnaeroGen™; Oxoid Ltd., Hampshire, England) were used for creating anaerobic conditions. Microcapsules containing live bacteria were homogenized manually to dilution and plating. Cell count was determined by anaerobic spread plate on MRS agar after 48 hours and was kept constant at 10¹⁰ cfu/g throughout the experiment.

Preparation of APA Microcapsules Loaded with L. acidophilus.

APA capsules were prepared aseptically using an Inotech Encapsulator™ IER-20 (Inotech Biosystems Intl. Inc. Switzerland) with a nozzle size of 300 μm at a frequency of 1160 Hz, 26.9 syringe pump speed and a voltage of 1.000 kV using a 60 ml syringe. 60 ml of 1.5% (w/v) sodium alginate (low viscosity) was mixed with 3 g of harvested bacterial cells (approximate cell load 10¹⁰ cfu/g) by centrifuging twice at 3000×g for 15 minutes with a single wash in 0.85% physiological solution between centrifugations. The formed microcapsules were hardened in 0.1M calcium chloride solution for 30 minutes, the optimal hardening time (Chandramouli, V. et al., (2004), Journal of microbiological methods 56:27-35). The resulting microcapsules were coated with 0.1% PLL and 0.1% alginate solution in the same manner as in preparation of APA microcapsules mentioned below. These APA microcapsules loaded with bacterial cells were washed twice with 0.85% physiological solution and stored at 4° C. until further use.

Preparation of Non-Loaded Apa Microcapsule.

APA capsules were prepared according to the standard protocol (Sun, A. M. F. et al., (1987), Crc Critical Reviews in Therapeutic Drug Carrier Systems 4:1-12) but with several modifications. Briefly, Ca-alginate beads were exposed to PLL solution (0.1% w/v) for 10 minutes washed twice with physiological solution (0.85% w/v, pH 7.2); finally put in alginate solution (low viscosity, 0.1% w/v) for 10 minutes. The resulting APA microcapsules were washed twice with 0.85% physiological solution and stored at 4° C. until used.

Microcapsule Mechanical Stability Test.

For mechanical stability evaluations, spherical (580±26 μm) APA membrane microcapsules were subjected to in vitro mechanical shaking incubation (150 rpm) in MRS broth for 76 hours in a Lab Line Environ Shaker at 37° C. Empty and L. acidophilus loaded APA microcapsules were also exposed to various test fluids: simulated gastric fluid (SGF) and simulated intestinal fluids (SIF), for 3, 12 and 24 hours at 150 rpm shaking and at 37° C. Samples were withdrawn and visually analyzed for physical damage using an optical light microscope.

Evaluation of Microencapsulated Live L. acidophilus Cells Viability in Yogurt.

Over a four-week study, we tested for the survival of encapsulated L. acidophilus cells in yogurt. The test samples contained 10 g of APA microcapsules loaded with L. acidophilus cells and 10 g of empty APA microcapsules, each immersed in 100 mL of yogurt. Two control samples were set up as follows: 1 g of APA microcapsules loaded with L. acidophilus cells in 10 mL of (0.85%, pH 7.2) physiological solution and 1 g of empty APA microcapsules in 10 mL of (0.85%, pH 7.2) physiological solution. The microcapsules were filled into 200 mL polyethylene wide mouth dilution tubes in which the bottoms were cut out and replaced with mesh net (200 microns) and placed into 2 L polyethylene containers. The microcapsules were trapped to ensure a proper separation from the bacterial cultures of L. acidophilus cells already present in the yogurt when purchased. Before microcapsules were analyzed for the viability of the encapsulated bacterial cells they were washed in (0.85%, pH 7.2) physiological solution 10 times to ensure complete removal of yogurt particulates. All the samples were stored at 4° C. and exposed to shaking at 100 rpm. Sampling was performed on a weekly basis and photomicrographs were taken at the same time.

Microcapsule Leakage Study

Microcapsule membrane leakage was monitored on a weekly basis by plating the 0.85% physiological solution in which the APA microcapsules loaded with L. acidophilus cells were stored for a period of 4 weeks at 4° C.

Evaluation of the Survival of Microencapsulated L. acidophilus Cells in Different pH Environments with and without Addition of Yogurt

To test for survival of cells in different GI pH environments, the following buffers were prepared: pH 2 of 0.2M KCl buffer, pH 3 of 0.1M KHP buffer and pH 4 of 1.0M KHP buffer, pH 6 of 0.1M KH₂PO₄ buffer and pH 8 of 0.1M TRIS buffer. For the experiments 400 mL of each buffer was autoclaved and cooled to room temperature and 100 mL of yogurt was added. The bottoms of 15 mL polyethylene tubes were. cut out and replaced with a 200 μm nylon mesh. These modified tubes were then filled up with 10 g of L. acidophilus loaded APA microcapsules. Samples were stored in anaerobic conditions at 37° C. in glass bottles. Sampling under sterile conditions was performed during the following time intervals: 5, 10, 15, 30, 60, 120, 180, 360, 1080, 2520 and 4320 minutes.

In addition, a second survival test was performed in which the above mentioned buffers were loaded with the same microcapsule bacterial load but without the addition of 2% M.F. yogurt. Samples were stored in anaerobic conditions at 37° C. in glass bottles. Sampling under sterile conditions was performed in the following time intervals: 30, 120, 180, 1080, 2520 and 4320 minutes.

Evaluation of Microencapsulated L. acidophilus Cells Survival in Human GI Model-Reactor Simulating the Stomach

Microcapsules containing live bacterial cultures L. acidophilus were tested using computer controlled simulated human GI model. In the model, each of the five reactor vessels represents distinct parts of the human GI tract in the following order: the stomach, the small intestine, the ascending colon, the transverse colon and the descending colon. In this experiment, 2 hour testing was performed in the first vessel representing the stomach in which a simulated gastric fluid (SGF), a carbohydrate-based diet was composed of arabinogalactan 1.0 g/L, pectin 2.0 g/L, xylan 1.0 g/L, starch 3.0 g/L, glucose 0.4 g/L, yeast extract 3.0 g/L, peptone 1.0 g/L, mucin 4.0 g/L, cysteine 0.5 g/L and pH was adjusted with 0.2N HCl was used. 1.5 g of APA microcapsules loaded with L. acidophilus was added to 10 mL of SGF fluid and 5 mL of yogurt. The control sample was SGF fluid. The study compared the survival of free L. acidophilus in SGF fluid only and APA microcapsules loaded with L. acidophilus in SGF fluid but in the absence of yogurt.

Statistical Methods

The Microsoft® Excel SP-2 software (Microsoft Corporation, USA) was used for all statistical analysis and the data are presented as mean and standard deviation.

Results

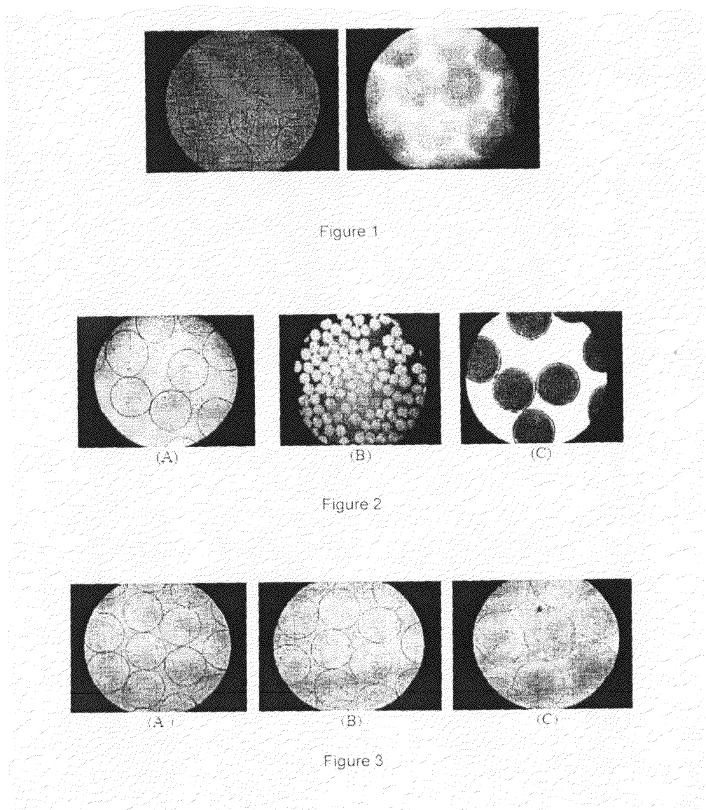

Formation of the APA Microcapsules In order to investigate the viability of L. acidophilus cells in various media, a microencapsulation procedure with specific parameters was followed which yielded spherical APA microcapsules of narrow size distribution and a constant bacterial cell load. FIG. 1 displays photomicrographs of freshly encapsulated empty capsules and capsules loaded with L. acidophilus cells. In the photomicrographs, under light microscopy, the capsules reveal their homogeneity, spherical shape and similar size. The empty APA microcapsules appear translucent and L. acidophilus loaded APA microcapsules are opaque owing to a dense load of L. acidophilus cells. Each subsequent microencapsulation yielded a similar bacterial cell load, kept constant at 10¹⁰ cfu/g.

Mechanical and GI Stability of APA Microcapsules

To assure viability of the L. acidophilus cells in the APA microcapsules, the microcapsules need to be resistant to mechanical stress. Empty and L. acidophilus loaded APA microcapsules were exposed to 200 rpm mechanical in vitro shaking for 76 hours in MRS broth at 37° C. FIG. 2 depicts photomicrographs of freshly prepared empty APA microcapsules as well as those loaded with L. acidophilus cells after an incubation period of 76 hours. A study of the APA capsule morphology revealed that no structural damage was visually noticeable; and therefore they were considered suitable for further testing.

An evaluation of the GI stability of APA microcapsule was carried out by exposing the APA microcapsules containing live LAB cells to simulated gastric fluid (SGF) solution (pH 1.98) at 37° C. for 3, 12 and 24 hours with 150 rpm mechanical shaking. Microscopic assessment was performed to evaluate microcapsule integrity. Results show that APA microcapsules were sturdy after exposure and remained intact in SGF. for up to 24 hours at pH 1.98 and with 150 rpm shaking (FIGS. 3a, 3b and 3c). We also evaluated the APA microcapsule stability in simulated intestinal fluid (SIF) at 37° C. and with 150 rpm mechanical shaking. The APA membrane was found to have remained intact and microcapsules shown to preserve their original spherical shape after 24 hours. APA microcapsules were seen to swell after 3 hours.

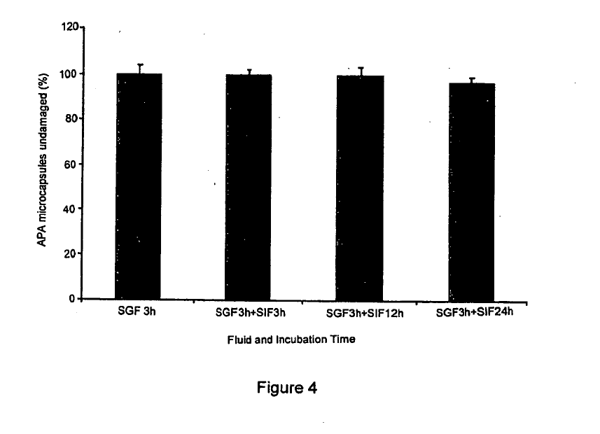

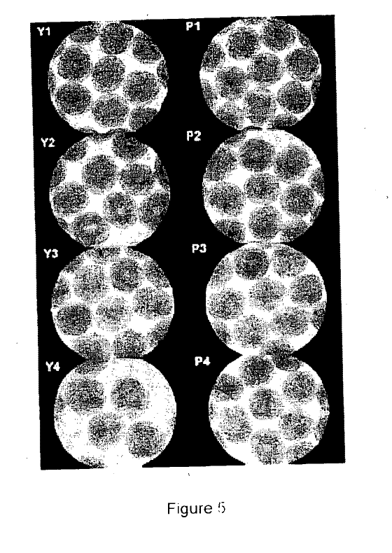

A comparative study wherein APA microcapsules were exposed to a combination of simulated fluids was also performed. FIG. 4 shows the percentage of undamaged APA microcapsules as a function of time; 100% of the APA microcapsules were unchanged after exposure to SGF for 3 hours and SIF for 3 hours. Moreover, no damage was found to occur to the APA microcapsules after treatment for 3 hours in SGF and 12 hours in SIF. However, up to 3% of the APA microcapsules were found to have been damaged after treatment in SGF for 3 hours and SIF for 24 hours.

The present invention will be more readily understood by referring to the following examples, which are given to illustrate the invention rather than to limit its scope.

### Example 1

### Microencapsulated L. acidophilus Cells Survival in Yogurt and Microcapsule Permeability Study

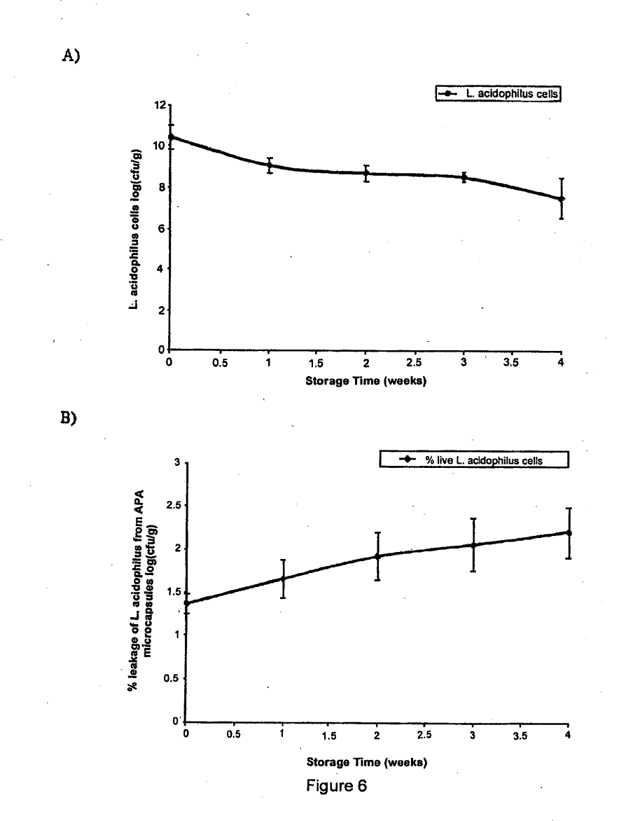

Studies were designed to investigate APA encapsulated bacterial cell survival in probiotic yogurt. FIG. 5 shows photomicrographs of APA microcapsules loaded with L. acidophilus cells. Pictures Y1 to Y4 were taken weekly over a period of 4 weeks and show APA microcapsules stored in 2% M.F. plain yogurt exposed to mechanical shaking at 100 rpm at 4° C. Photomicrographs P1 to P4 show APA microcapsules stored in 0.85%, physiological solution, over 4 weeks, stored under similar conditions of 4° C. and shaking at 100 rpm. This 4-week study revealed that APA microcapsules loaded with L. acidophilus cells preserve their shape and integrity over time. The survival of encapsulated L. acidophilus over the 4-week study is shown in FIG. 6(A). There was a constant drop observed in bacterial cell survival and it reached 7.53 log cfu/g of live bacterial cells after the fourth week of testing. This is however, a rather acceptable loss considering the cell count decreased from 10¹⁰ cfu/g to 10⁷ cfu/g, which is usually the minimum requirement for a yogurt to be labeled probiotic. In addition, the mean pH of the yogurt stored at 4° C. measured on a weekly basis was found to be 4.3.

The capacity of APA microcapsules to retain its cell load was measured over 4 weeks. APA microcapsules loaded with L. acidophilus cells were stored in 0.85% physiological solution at 4° C. and the supernatant from the medium was plated weekly. FIG. 6(B) shows the percentage survival of live L. acidophilus cells in 0.85% physiological solution over time. A steady increase in the bacterial count was found over 4-weeks. After the fourth week, it was found that 2.21 log cfu/g of L. acidophilus cells had seeped from the APA microcapsules into the storage medium.

Survival of Microencapsulated L. acidophilus Cells in Different pH Environments with and without Supplementation with Yogurt

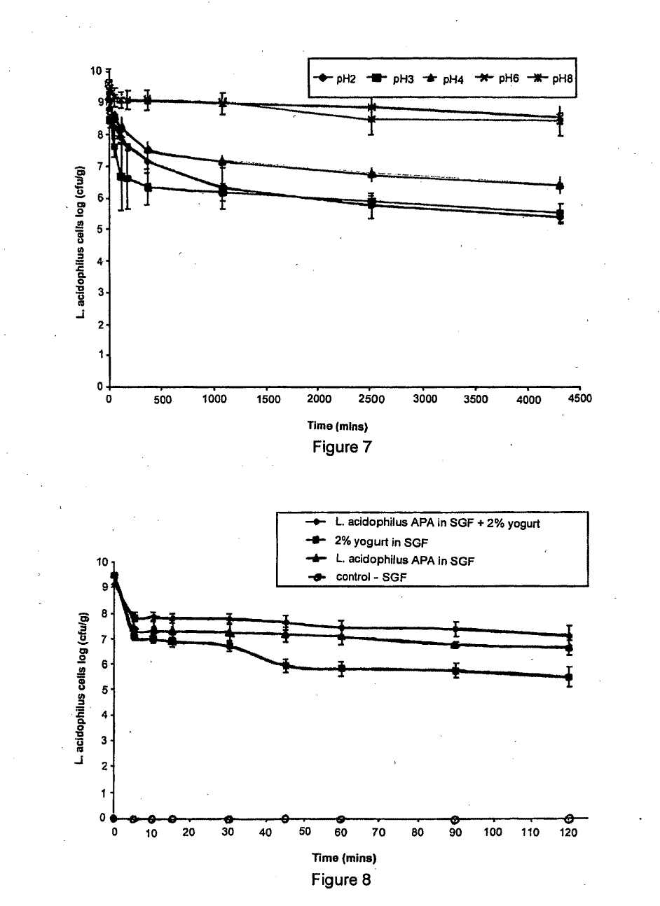

The survival of APA microcapsules loaded with L. acidophilus cells, in various environments, was estimated using a series of different buffers. The viability of encapsulated L. acidophilus cells in the presence of 2% M. F. yogurt in a buffer was tested over 72 hours (FIG. 7). Crucial time points at 120 minutes—the stomach's approximate retention time, and at 360 minutes—the small intestine's retention time showed 6.67 log cfu/g survival at pH 2 and 9.18 log cfu/g viability at pH 6, respectively. As expected, the lowest survival rates were found at the most acidic pH of 2 (5.38 log cfu/g) and at pH 3 (5.52 log cfu/g) after 72 hours. At pH 6 (representative of the small intestine) the viability was seen to be 8.43 log cfu/g after 72 hours, and 6.41 log cfu/g at pH 4. While there was a steep drop in the total bacterial count during the first 3 hours, a slower decline was observed from the 6th hour onward until the 72nd hour. While the L. acidophilus bacterial cultures survived at pH 6 and pH 8, there were no viable cells present at lower pH values beyond the 30 minutes sampling time interval.

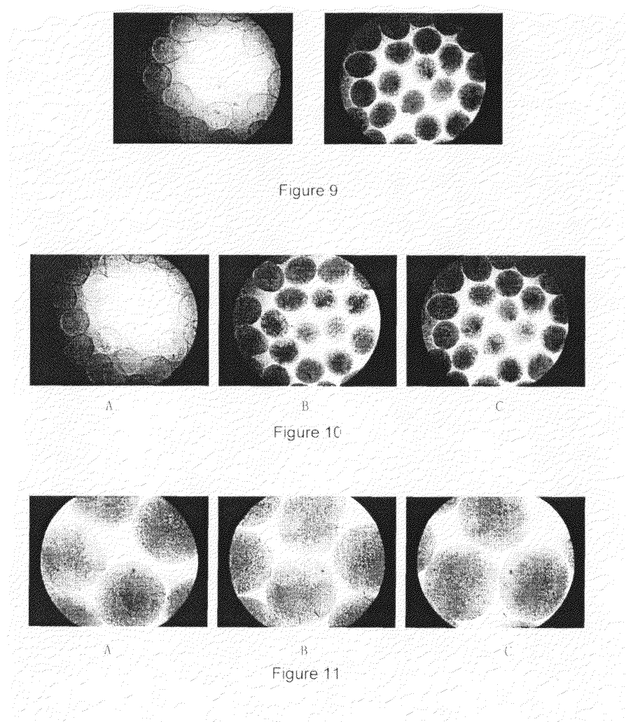

Using a computed controlled simulated model of the human GI tract, a study of the survival of encapsulated bacteria under gastric conditions of pH 1.98 was carried out. Three samples were used; the first, encapsulated L. acidophilus cells in SGF; second, encapsulated L. acidophilus cells in SGF and 2% M. F. yogurt and the third, 2% M. F. yogurt containing free bacterial cultures in SGF. An SGF sample served as a control. During the stomach's 2-hour retention time, the anaerobic survival of L. acidophilus cells at 37° C. was determined. As shown in FIG. 8, the lowest bacterial count was obtained in the sample containing 2% M. F. yogurt with free bacterial cells in SGF. The highest survival (7.10 log cfu/g) was observed in the sample containing encapsulated L. acidophilus cells in presence of 2% M. F. yogurt and SGF. A slightly lesser survival (6.66 log cfu/g) was determined in the sample containing encapsulated L. acidophilus cells in SGF.

A novel yogurt formulation for oral bacterial delivery using microencapsulation technology was designed in accordance with the present invention. The probiotic bacterium L. acidophilus was encapsulated within APA microcapsule. Any matrix for cell immobilization ideally should provide physical support and uniform distribution of immobilized cells where the transport gradient of nutrients toward and waste products away is balanced and necrosis is prevented. In past studies, it has been reported that the most common type of membrane used for cell therapy is the single alginate based polymer membrane. Various other substances are also being used for encapsulation such as various proteins, polyhemoglobin, and lipids. From a variety of naturally derived membrane materials (e.g. pectin, chitosan, hydroxyethyl methacrylate (HEMA), agarose and lipid complexes), the alginate and poly-L-lysine capsule was selected because alginate is an accepted, generally regarded as safe (GRAS) non-toxic food additive and poly-L-lysine is a natural, safe poly-aminoacid. Calcium ions provide cross-linking with sodium alginate through ionotropic gelation. The PLL coating is shown to provide immunoisolation. The outer alginate layer coating the microcapsules provides better acid stability and improved mechanical strength. In doing so, the biocompatibility of the multilayer structure is optimized. The molecular weight cut off (MWCO) of the resultant APA membrane was determined to be 60-70 KD, which provides a useful selectivity limit. This would allow the polymer membrane to protect encapsulated materials from harsh external environments, while at the same time allowing for the metabolism of selected solutes capable of passing in and out of the microcapsule.

The microencapsulation technique used yields spherical alginate microcapsules that have a narrow size distribution and retain L. acidophilus bacterial cultures (FIG. 1). To be suitable for oral delivery, microcapsules must demonstrate good mechanical resistance and results show that the APA microcapsules maintain their integrity even after prolonged mechanical agitation (FIG. 2). In addition, the APA microcapsules demonstrated excellent resistance to simulated intestinal and gastric fluids and only underwent a slight swelling when exposed to SGF for 3 hours and SIF for 24 hours at 37° C. with agitation at 150 rpm (FIG. 3). As shown in FIG. 4, 97% of the microcapsules remain intact after being exposed for 3 hours to SGF and 24 hours to SIF at 150 rpm and 37° C. Microencapsulated L. acidophilus cells were later stored in 2% M.F. yogurt and physiological solution (0.85%, pH 7.2) over 4 weeks. The viability of live L. acidophilus in microcapsules and their morphology was monitored. From the photomicrographs, taken weekly, it is seen that the shape of the microcapsules is well preserved and when compared to microcapsules stored in physiological solution, neither the 2% M.F. yogurt nor shaking at 100 rpm alters their integrity or appearance (FIG. 5). Both media, differing significantly in their viscosities (2% M. F. yogurt and 0.85% physiological solution) serve equally well as storage media for APA microcapsules. This implies superior resistance to mechanical shear and a tolerance to the various components of the simulated GI fluids.

An initial cell load of 10⁷ cfu/g is recommended by National Yogurt Association for yogurt to be called a probiotic. These high numbers have been suggested to compensate for the possible loss in the numbers of probiotic organisms during passage through the stomach and intestine. In our studies, a cell load of 10¹⁰ (cfu/g) was used. Higher initial load was selected to ensure delivery of a greater number of live bacteria to target sites.

Over 4 weeks storage, 7.53 log (cfu/g) of the encapsulated bacteria remained alive with 100 rpm shaking at 4° C. (FIG. 6(A)). This duration was chosen as it approximates the length of time yogurt can be stored in a refrigerator after purchase. The microcapsule permeability study performed over 4 weeks shows a steady release of the bacteria into the physiological storage solution (0.85% NaCl, pH 7.2). The cumulative count after 4 weeks was found to be approximately 2.21 log (cfu/g) of the encapsulated live bacteria (FIG. 6(B)). Thus the microcapsules seem to retain bacteria adequately.

Bacterial cells encounter a variety of pH's during the period of their GI transit. The ability to resist and to adapt to these changes is a desirable property in a probiotic. Buffers of various pH values were prepared to mimic the conditions microencapsulated L. acidophilus cells might encounter during passage in the GI tract. A comparative study was also performed in presence and in absence of 2% M.F. yogurt. FIG. 7 shows the survival rates of microencapsulated L. acidophilus at different pH's in presence of 2% M.F. yogurt.

The survival rates of microencapsulated L. acidophilus in the same conditions without the addition of yogurt were much lower. As expected, after a 72-hour period, the survival at pH 2 was 5.38 log (cfu/g), at pH 3 was 5.52 log (cfu/g), at pH 4 was 6.41 log (cfu/g), at pH 6 was 8.44 log (cfu/g) and at pH 8 was 8.55 log (cfu/g). This shows a lower tolerance and consequently higher sensitivity to extremely acidic environments. As prior results have shown, gastric fluids are detrimental to probiotic cell counts (Lee and Heo, 2000). It seems however, that encapsulated cells survive gastric conditions significantly better when stored in yogurt as opposed to storage in media sans yogurt.

The survival of free and encapsulated L. acidophilus cells in SGF and in the presence and absence of 2% M.F. yogurt was estimated. FIG. 8 shows the survival of encapsulated and free bacteria using a model of a human stomach at 37° C. over two hours, the time it takes food to pass through the stomach. After two hours, 7.10 log (cfu/g) of microencapsulated L. acidophilus cells in the presence of SGF and 2% M.F. yogurt were still alive, while only 5.51 log (cfu/g) of free L. acidophilus cells were found to be viable in presence of SGF. In addition, 6.66 log (cfu/g) of microencapsulated L. acidophilus cells in SGF fluid without yogurt were reported alive. The difference in the survival of encapsulated and free bacterial cells could thus, as predicted, be attributed to the protective effect of the APA membrane in the presence of SGF. Moreover, the addition of 2% M. F. yogurt indicates that yogurt might posses some additional protective properties. The protective effect of yogurt on bacterial cells has been attributed to several factors. These include the strains of inherent probiotic bacteria, pH, hydrogen peroxide, storage atmosphere, concentration of metabolites such as lactic acid and acetic acids, dissolved oxygen, and buffers such as whey proteins (Dave, R. I. et al., (1997), International Dairy Journal 7:31-41; Kailasapathy, K et al., (1997), Australian Journal of Dairy Technology 52:28-35).

The difference in the survival of microencapsulated L. acidophilus cells in the presence and absence of 2% M.F. yogurt indicates that yogurt may further help protect microencapsulated L. acidophilus cells.

Results show that APA microcapsules display good mechanical stability in storage solutions. This study also demonstrates the protective properties of the APA membrane in low pH conditions, and in simulated gastric fluid. This indicates that ingested microcapsules may be capable of surviving the passage through the stomach and reaching the target sites further in the GI tract with an adequate cell load which can be further enhanced by using yogurt. In vitro result suggests that yogurt containing APA microencapsulated L. acidophilus may represent a significant improvement over ordinary yogurt in the delivery of probiotic bacterial cells for possible treatment of GI tract related diseases such as in colon cancer. Further studies, however, are required to substantiate this hypothesis, in particular in vivo confirmation of their effectiveness in experimental animal models.

### Example 2

### Materials and Method

Sodium alginate (low viscosity), poly-1-lysine (MW=27,400) (lot 71K5120) and calcium chloride (desiccant, 96+%, A.C.S. reagent, FW 110.99, d 2.15, batch # 05614AC) were purchased from Sigma-Aldrich, Canada. MRS AGAR Difco™ Lactobacilli and MRS BROTH Difco Lactobacilli were purchased from Becton, Dickinson and Company Sparks, USA. Chitosan 10 was from Wako Chemicals, Japan. Liberty plain yogurt 0% and 2% M. F. containing active Acidophilus and Bifidus cultures was procured from a local store.

Bacteria Cultures, Propagation and Enumeration

L. acidophilus (ATCC 314) cells were inoculated in 100 mL of MRS broth. The bacteria were cultured in MRS Broth at 37° C. in a Professional Sanyo MCO-18M Multi-Gas Incubator. Cultures were grown for 24 hours and centrifuged at 3000×g for 15 minutes at 37° C. The media was decanted; the cells were suspended in 100 mL of fresh MRS media and incubated for an additional 20 hours at 37° C. After growth was performed, the resulting cell wet weights were noted. Anaerobic jars and gas generating kits (Atmosphere Generation System AnaeroGen™; Oxoid Ltd., Hampshire, England) were used for creating anaerobic conditions. Microcapsules containing live bacteria were homogenized manually to dilution and plating. Cell count was determined by anaerobic spread plate on MRS agar after 48 hours and was kept constant at 10¹⁰ cfu/g throughout the experiment.

Preparation of Ac Microcapsules Loaded with L. acidophilus

Alginate-Chitosan (AC) microcapsules were prepared aseptically using an Inotech Encapsulator® IER-20 (Inotech Biosystems Intl. Inc. Switzerland) in a Microzone Biological Containment Hood (Microzone Corporation ON, Canada). The following parameters for microencapsulation were used: a nozzle size of 300 μm at a frequency of 918 Hz, 24 syringe pump speed and a voltage of >1.000 kV using a 60 ml syringe. All membrane components were filter sterilized through a 0.22 μm Sterivex-GS filter prior to use. The pellet of wet cells was centrifuged twice at 3000×g for 10 minutes, weighted and kept constant at 1.7 g, suspended in 0.85% saline, pooled and slowly added to a gently stirred 60 mL sterile 1.5% (w/v) sodium alginate (low viscosity) solution. The approximate cell load was kept constant at 10¹⁰ cfu/g. Formed microcapsules were hardened in 0.1M calcium chloride solution for 30 minutes, the optimal hardening time. The resulting microcapsules were coated with 0.5%, 0.25% and 0.1% chitosan 10 solution dissolved in dilute acetic acid at a pH of 5.3 for 30 min. These AC microcapsules loaded with bacterial cells were washed twice with 0.85% physiological solution and stored at 4° C. until further use.

Preparation of Non-Loaded AC Microcapsule

AC capsules were prepared according to the standard protocol with several modifications. Briefly, Ca-alginate beads were exposed to chitosan solution (0.5% w/v) for 30 minutes, washed twice with physiological solution (0.85% w/v, pH 7.2). The resulting AC microcapsules were washed twice with 0.85% physiological solution and stored at 4° C. until used.

Preparation of AC Microcapsules

The feasibility of delivering the encapsulated live bacterial cells to various compartments of GI model was studied. For this, spherical AC microcapsules of narrow size distribution and a constant bacterial cell load were prepared. FIG. 9 displays photomicrographs of freshly encapsulated empty capsules and capsules loaded with L. acidophilus cells of 550±26 μm in size and magnification of 2.5× using light microscopy. Microcapsules exhibit homogeneous spherical shape. Empty capsules are transparent and capsules loaded with bacterial cells are opaque due to high concentration. Each subsequent microencapsulation yielded a similar bacterial cell load, kept constant at 10¹⁰ cfu/mL.

Mechanical and GI stability of AC Microcapsules

In order to test microcapsules mechanical stability freshly prepared capsules were subjected to mechanical stress. FIG. (10a) displays empty AC microcapsules, FIG. (10b)—loaded capsules with L. acidophilus and FIG. (10c), same capsules after 76 hours of incubation in MRS broth exposed to mechanical shaking of 150 rpm at 37° C. It can be observed that the physical morphology of the capsules after being subjected to an intense mechanical stress does not impact capsules integrity or their shape. Upon close examination no damage was noted. Therefore, the capsules preserve their robustness while being exposed to harsh conditions.

Having obtained promising results from physical testing, microcapsules containing bacterial cells were subjected to various fluids found in SHIME (FIG. 11). In addition, all the samples were exposed to mechanical shaking of 150 rpm at 37° C. All the photomicrographs were taken using magnification of 6.3×. Upon close examination no physical damage was observed and the capsules remained intact.

In FIG. 12, the bars shows 97.42%, 91.11%, 88.43% and 84.19% integrity of AC capsules treated in SGF for 3 hours, SGF and SIF for 3 hours, SGF 3 hours followed by 12 hours in SIF and SGF 3 hours followed by 24 hours in SIF, respectively. As in previous studies, the microcapsules were exposed to mechanical shaking of 150 rpm at 37° C.

Evaluation of Bacterial Survival in Human GI Model-Reactor Simulating the Stomach

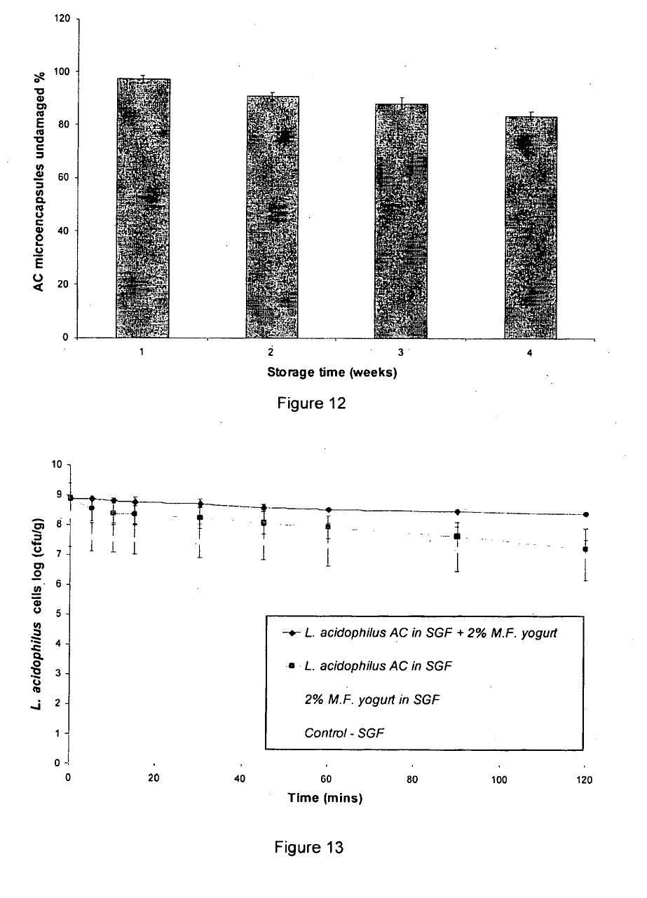

Studies were designed to investigate AC encapsulated bacterial cell survival in probiotic yogurt. Firstly, we compared the survival of encapsulated bacterial cells in SGF with and without addition of 2% M.F. yogurt as well as the survival of free bacteria contained in the yogurt. FIG. 13 shows the predicted results. We anticipated the highest survival rate of AC encapsulated cells in presence of yogurt, which after 120 min. yielded 8.37 log cfu/mL. 7.20 log cfu/mL of viable cells was found of encapsulated L. acidophilus and 7.00 log cfu/g of free bacteria treated with SGF for 120 min.

Evaluation of Bacterial Survival in Human GI Model-Reactor Simulating the Small Intestines

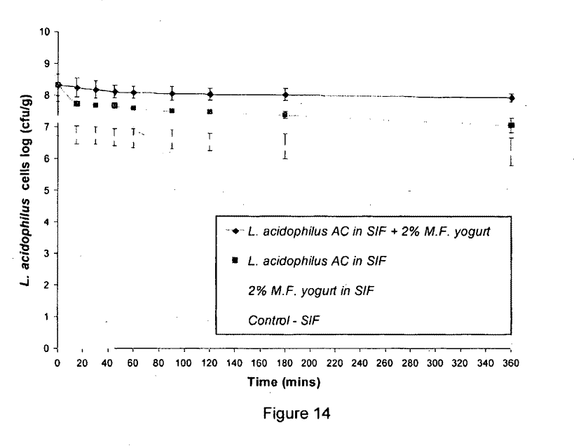

FIG. 14 displays survival of AC encapsulated and free bacterial cells obtained by exposure to simulated intestinal fluid conditions. The viability of encapsulated L. acidophilus and free cells in the presence and absence of 2% M. F. yogurt was tested over 6 hours. Crucial time points at 120 minutes—the stomach's approximate retention time, and at 360 minutes—the small intestine's retention time showed 8.05, 7.47, 6.54 and 7.96, 7.09 and 6.24 log cfu/mL, respectively.

Evaluation of AC Microencapsulated live L. acidophilus Cells Viability in Yogurt

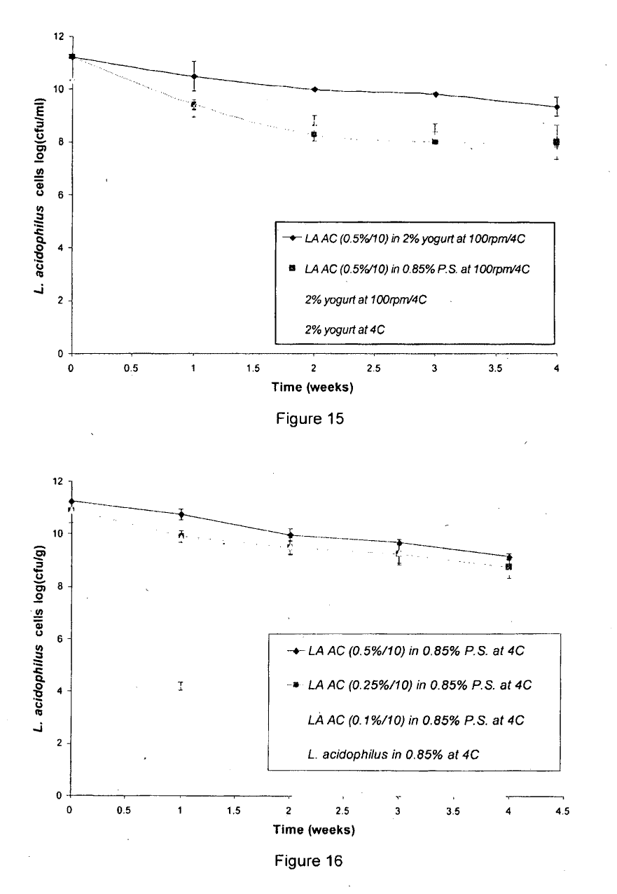

Comparative study measuring the performance of AC microcapsules in yogurt, using chitosan 10, was performed over 4 weeks. AC microcapsules loaded with L. acidophilus cells were stored in 2% M.F. yogurt and 0.85% physiological solution at 4° C. and exposed to mechanical shaking of 100 rpm (FIG. 15). There was a constant drop observed in bacterial cell survival and it reached 9.37 log cfu/mL of cells encapsulated with chitosan 10 and stored in yogurt, 8.04 log cfu/mL of cells encapsulated with chitosan 10 and stored in physiological solution at the fourth week of testing. All the samples were exposed to mechanical shaking of 100 rpm and stored at 4° C. In addition, free bacterial cells contained in the yogurt decreased their count to 7.65 log cfu/mL and 8.26 log cfu/mL for yogurt exposed to mechanical shaking of 100 rpm and yogurt stored at 4° C., respectively.

FIG. 16 depicts a study performed during 4 weeks where different chitosan 10 concentrations were used, namely 0.5%, 0.25% and 0.1%. Microcapsules coated with these polymers were stored in 0.85% physiological solution and kept at 4° C. Free L. acidophilus cells in 0.85% physiological solution were set up as a control at 4° C. A constant drop of bacterial survival was observed over the 4-week study (FIG. 16). The highest survival rate was noticed for chitosan 10 at 0.5% concentration, 9.11 log cfu/mL and the lowest for chitosan 10 at 0.1% concentration-8.56 log cfu/mL. Free bacterial cells have reached complete downfall at the second week.

Evaluation of the Survival of Microencapsulated and Free L. acidophilus Cells in Different pH Environments with and without Addition of Yogurt

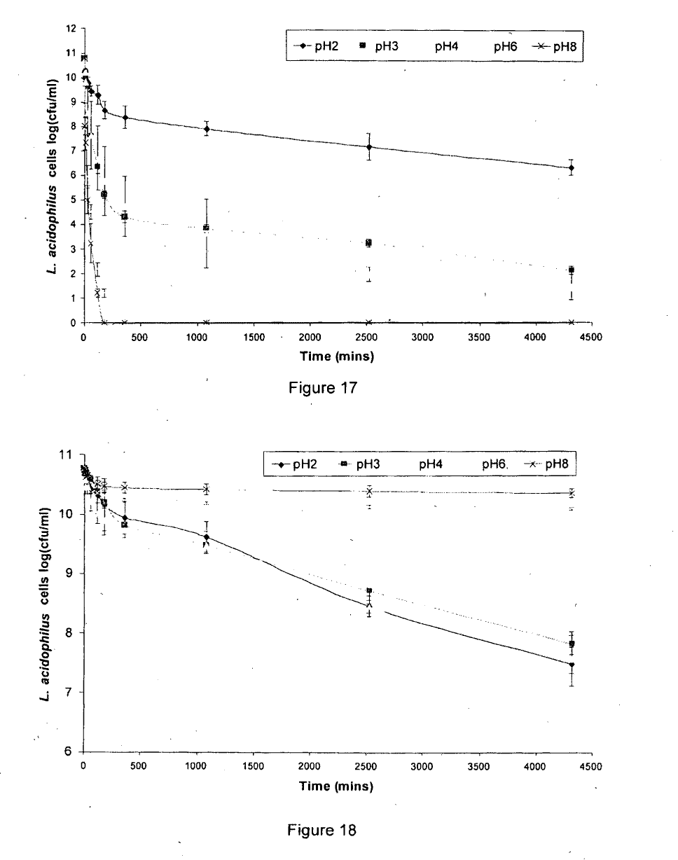

Another set of experiments was designed to compare the viability of free L. acidophilus cells in 2% M.F. plain yogurt in various buffers with the viability of AC encapsulated bacterial cells during 72-hour study. FIGS. 17 and 18 show the results. In FIG. 17, the highest survival of 6.34 log cfu/mL was read for free L. acidophilus cells at pH2, followed by 2.14 log cfu/mL and 1.44 log cfu/mL at pH3 and 4, respectively. When the cells were exposed to pH6 and 8, they entirely decreased after 360 and 180 min, respectively. On the other hand, bacterial cells did not show complete decline when AC encapsulated and exposed to the same conditions. FIG. 18 shows the survival of encapsulated live L. acidophilus cells in buffers of pH 2, 3, 4, 6 and 8 supplemented with 2% M.F. yogurt. Contrary to the previous results, cells exhibited the highest survival at pH 8, 10.34 log cfu/mL, and lowest at pH2 of 7.48 log cfu/mL. Moreover, at pH 6 cells reached 10.07 log cfu/mL, at pH 4, 7.56 log cfu/mL and 7.82 log cfu/mL at pH 3. This is consistent with the lactic acid bacteria as they produce lactic acid as a result of carbohydrate fermentation and their growth lowers both the carbohydrate content of the media that they ferment, and the pH due to lactic acid production.

### Example 3

### Materials and Methods

Sodium alginate (low viscosity), poly-L-lysine (MW=27,400) (lot 71K5120) and calcium chloride (desiccant, 96+%, A.C.S. reagent, FW 110.99, d 2.15, batch # 05614AC) were purchased from Sigma-Aldrich, Canada. MRS AGAR Difco™ Lactobacilli and MRS BROTH Difco™ Lactobacilli were purchased from Becton, Dickinson and Company Sparks, USA. Liberty plain yogurt 2% M. F. containing active Acidophilus and Bifidus cultures was procured from a local store.

Lactobacillus acidophilus (ATCC 314) cells were cultivated and serially propagated three times in the MRS medium before experimental use. Incubations were performed at 37° C. in a Professional Sanyo MCO-18M Multi-Gas Incubator in anaerobic conditions (1-2% CO₂, Atmosphere Generation System AnaeroGen™; Oxoid Ltd., Hampshire, England). Bacteria to be encapsulated were isolated after 20 hours of the 3^{rd} passage.

Microencapsulation Method

The bacterial strains were microencapsulated into Alginate-Poly-L-Lysine-Alginate (APA) membranes. All membrane components were filter sterilized through a 0.22 μm Sterivex-GS filter prior to use. Grown cultures were centrifuged at 3000×g for 15 minutes at 25° C. and the supernatant broth was decanted. The pellet of wet cells was weighted and suspended in 0.85% saline, pooled and slowly added to a gently stirred sterile 3.3% sodium alginate solution (diluted 50% with 0.85% saline). The entire procedure was performed under sterile conditions in a Microzone Biological Containment Hood (Microzone Corporation ON, Canada) and all solutions were autoclaved with the exception poly-L-lysine which was 0.22 μm sterile-filtered prior to usage. APA microcapsules were prepared aseptically using an Inotech Encapsulator® IER-20 (Inotech Biosystems Intl. Inc. Switzerland). Freshly prepared microcapsules were washed twice with 0.85% saline and stored at 4° C. Parameters for microencapsulation were as follows:

TABLE 1
Microencapsulator settings

| Parameter | Setting |
|---|---|
| Final Alginate Concentration | 1.65% |
| Cell Load | 2.5 g/60 mL Alginate |
| Gelation time in CaCl₂ | 30 min |
| Coating materials | 0.1% PLL, 0.1% Alginate |
| Coating time | 10 min each |
| Nozzle Diameter | 300 μm |
| Vibrational Frequency | 918 Hz |
| Syringe Pump speed | 24.0 |
| Voltage | >1.00 kV |
| Current | 2 amp |

Treatment Formulation Preparation

APA microcapsules loaded with L. acidophilus bacterial cells were blended with Liberty plain yogurt 2% M.F. and 0.85% saline in the proportions of 1.5:0.5, respectively. Empty APA microcapsules were suspended in 0.85% saline using same formulation.

In Vivo Mouse Colorectal Cancer Model.

Multiple intestinal neoplasia (Min) mice are heterozygous for ApcMin+(Min), a germ-line truncating mutation at codon 850 of. the Apc gene and spontaneously develop pretumoric numerous intestinal neoplasms⁴¹. The Apc (Min/+) mouse is a popular animal model for studies on human colorectal cancer⁴². It is used to study the effects of genetics, diet, or chemical compounds on the incidence and development of intestinal precancerous lesions, the adenomas⁴³. The germ-line mutations in the APC gene lead to FAP, but inactivation of APC is also found in 80% of sporadic colorectal cancers⁴⁴.

Male heterozygous C57BL/6J-Apc^{Min/+} mice, weighing 20-25 g, were obtained from The Jackson Laboratory (Bar Harbor, Me.). The animals kept in the Duff Medical Building Animal Care Facility on a 12-hour light-dark cycle and controlled humidity and temperature. They were allowed sterile water and the laboratory rodent diet 5001 from Purina Land O'Lakes ad libitum. Animals overall health was monitored daily.

The protocol was approved by the Animal Care Committee of McGill University and animals were cared for in accord with the Canadian Council on Animal Care (CCAC) guidelines.

Animal Protocol

Mice 7 or 8 weeks old were used. The life span of these mice is 119±31 days⁴⁶. The mice were separated into three experimental groups: 1) Control—animals were gavaged empty APA microcapsules suspended in 0.85% saline, 2) Treatment 1—animals were gavaged APA microencapsulated L. acidophilus bacterial cells blended in 2% M.F. yogurt and 3) Treatment 2—gavaged APA microencapsulated L. acidophilus bacterial cells suspended in 0.85% saline. Upon arrival, animals were randomly placed in the cages and allowed one week of acclimatization period. Based on initial serum IL-6 values the animals were ranked and randomly block assigned to the aforementioned groups. There were 11 animals per group. Animals were weighed individually every week; the saphenous vein was bleed every 4 weeks and feces samples were collected at specific intervals throughout the experiment. There were 3 end points at weeks 8, 10 and 12 of treatment at which 9, 9 and 15 animals were sacrificed, respectively.

Analytical Techniques.

Interleukin-6 Determination. Interleukin-6 (IL-6) is a cytokine secreted by diverse cell types under homeostatic and inflammatory conditions⁴⁷. Interleukin (IL)-6 mRNA expression in general is low in normal, adenomatous and cancerious human colon mucosa; except in rather undifferentiated lesions, in which IL-6 is over expressed. IL-6 has been shown to be associated with cancer development. However, its role in gastric cancer has never been investigated.

For this, blood samples were collected every 4 weeks into heperinized tubes which after blood collection were centrifuged at 5000×g for 20 minutes to yield plasma which was used in further testing. The release of IL-6 from plasma samples into the culture medium was quantified by enzyme-linked mouse immunosorbent assay (ELISA, Biosource, Invitrogen, USA) according to manufacturer's instructions. Briefly, 50 ul plasma plus 50 μL standard diluent buffer were added to each well and incubated for 3 hours and 30 minutes at room temperature. Upon completion of the assay procedure, the plate was read at 450 nm wavelength using a Perkin Elmer Victor microtiter plate reader.

The Hemoccult SENSA test. Used according to Beckman Coulter instructions. Briefly, using applicator provided small fecal sample were collected, thin smear was applied covering Box A. Second applicator was used to obtain second sample from a different part of feces, covering Box B. Three days later, samples were developed by applying one drop of Hemoccult SENSA Developer between the positive and negative Performance Monitor areas. Results were read within 10 seconds.

Fecal Bile Acids Determination. Feces were collected at specific intervals throughout the experiment and the analysis was performed per group per cage. Total fecal bile acids were determined as previously described^{48, 49} with the following modifications. 25 uL of sample were used to determine total bile acid concentration enzymatically as previously described⁵⁰ using a commercially available kit (Sigma Diagnostic Bile Acids 450A, Sigma Diagnostics, St. Louis, Mo., USA).

Adenoma Enumeration, Classification and Histopathology. The mice were euthanised by CO₂ asphyxiation, and the small, large intestine and cecum were excised. Upon removal, the intestines were infused with 10% Phosphate Buffered Formalin (PBF) after which the Swiss Roll was performed by which they were placed in cassettes and immersed in 10% PBF as a fixative. Five-um paraffin-embedded sections were stained with H&E for histological evaluation.

Polyp scoring was performed by a blinded veterinary pathologist to the treatment. The lesions observed were divided into two categories mostly based on the size of the lesion: gastrointestinal intraepithelial neoplasia (GIN)(<1 mm) and adenoma (>1 mm).

Statistical Analyses.

The Statistical Analysis System (SAS Enterprise Guide 4.1 (4.1.0.471) by SAS Institute Inc., Cary, N.C., USA) was used to analyze the data. Data were expressed as means±SEM. Differences in body weight, IL-6 concentration, adenoma and gastrointestinal intraepithelial neoplasia number between the groups were analyzed statistically by ANOVA Mixed Models. Data were considered significant at P<0.05.

Microencapsulation and Evaluation of the Treatment Impact on Min Mice Body Weight.

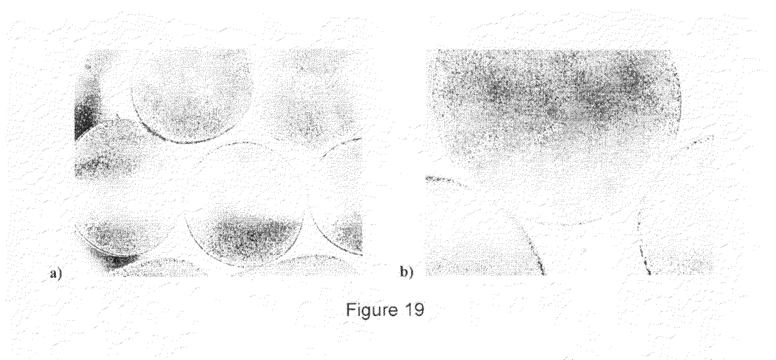

The microencapsulation technique used yielded spherical alginate microcapsules that have a narrow size distribution and retain L. acidophilus bacterial cultures. FIG. 19 displays photomicrographs of freshly encapsulated loaded with L. acidophilus bacterial cells capsules obtained using a light microscopy magnification of 6.3×. They were 433±67 μm in size. Using optimal settings each microencapsulation yielded a fixed bacterial cell load, kept constant in a range of 10¹⁰ cfu/mL.

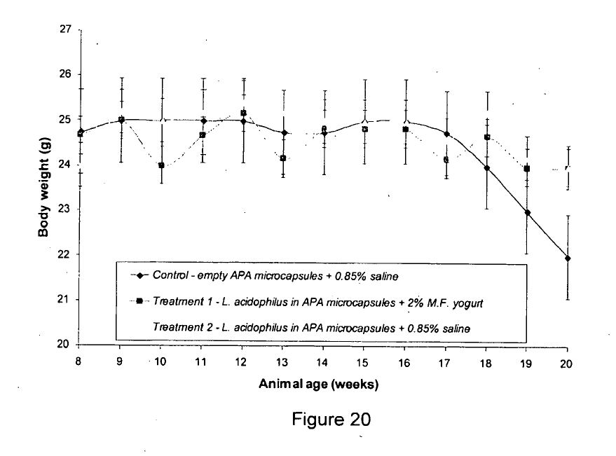

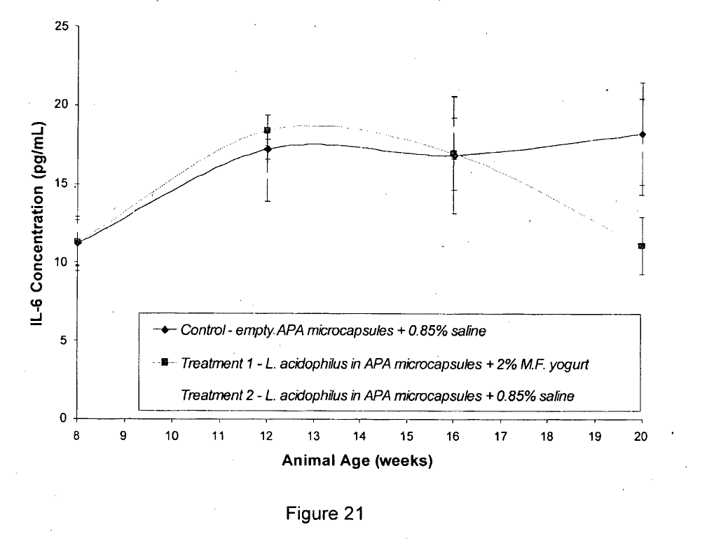

After acclimatization period of one week, the animals were randomly block assigned into 3 groups, each composed of 11 animals. Body weights were taken down on weekly basis. (FIG. 20). There was a steady drop of body weight in control group animals, from 24.6±0.48 to 22±1.47 grams over 12 weeks whereas the body weights remained stable in both treatment groups.

Interleukin-6 Level was Determined in Experimental Animals.

Results show the average levels of anti-inflammatory interleukin-6 was 11.17±1.59 for treatment 1 group, 17.45±2.74 for treatment 2 group and 18.33±1.46 μg/mL for the control group at the time of sacrifice (FIG. 21). The expression levels kept increasing in animals of control group and decreasing after the 16^{th} week of age in animals of both treatment groups.

Detection of Fecal Blood Presence in Min Mice Using the Hemoccult SENSA Test.

The feces samples from individual cages were collected at the beginning and end of the experiment. The occult blood test was performed in triplicates. All tests were positive and the coloration intensities from “+” being the least intense to “+++” being the most intense, were qualitatively scored by three observers. The results are displayed in the Table 2. The Hemoccult SENSA test results in a blue-colored compound which occurs when guaiac is oxidized by hydrogen peroxide. The abnormal bleeding is associated with gastrointestinal disorders and can be qualitatively detected with a higher sensitivity than standard guaiac tests.

TABLE 2
The Hemoccult SENSA test

| | Animal age - 8 weeks | | | Animal age - 20 weeks | | |
|---|---|---|---|---|---|---|
| | Cage | Cage | | Cage | | |
| | 1-3 | 4-6 | Cage 7-9 | 1-3 | Cage 4-6 | Cage 7-9 |
| Control | + | + | ++ | +++ | +++ | +++ |
| Treatment 1 | + | ++ | + | ++ | +++ | ++ |
| Treatment 2 | ++ | + | + | ++ | ++ | ++ |

Fecal Bile Acids Levels in Experimental Animal Model.

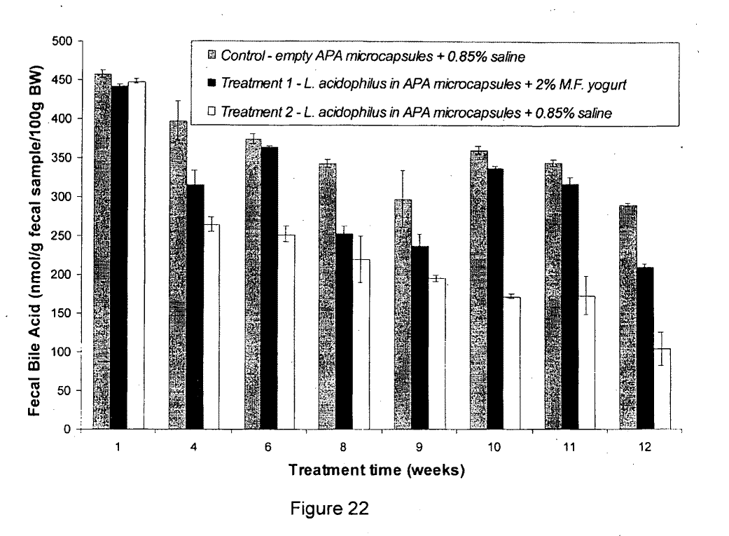

To determine the effect of microencapsulated probiotic bacteria on luminal bile acids we measured the levels of bile acids in samples of feces from individual cages of each group. There was a constant drop observed in all groups (FIG. 22). The greatest drop of 448±2.82 to 105±21.36 (nmol/g fecal sample/100 gBW) was observed in a group of treatment where animals were gavaged L. acidophilus bacterial cells in APA microcapsules+0.85% saline. A decrease of 442±4.87 to 210±3.66 was observed in a treatment group receiving microencapsulated L. acidophilus bacterial cells in 2% M.F. yogurt. The averaged total fecal bile acid values with their respective groups and p values are presented in Table 3.

TABLE 3
Comparison of average total fecal bile
acid per group and their p values.

| | Average Total Fecal | Repeated measures ANOVA |
|---|---|---|
| | Bile acid (nmol/g fecal | using Mixed Models |
| | sample/100 g BW) | Analysis p values |
| Control | 358.44 ± 53.93 | 0.0296 T1 + C |
| Treatment 1 | 310.25 ± 75.22 | 0.0187 T1 + T2 |
| Treatment 2 | 229.15 ± 101.95 | 0.0037 T2 + C |

Adenoma Reduction in the Treated Animals: Classification and Histopathology.

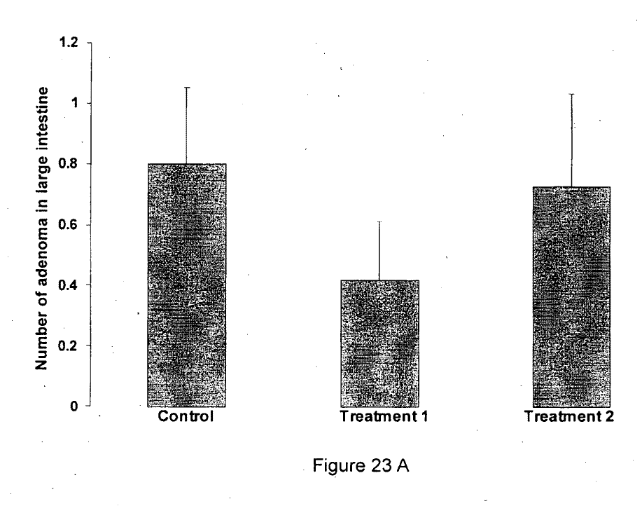

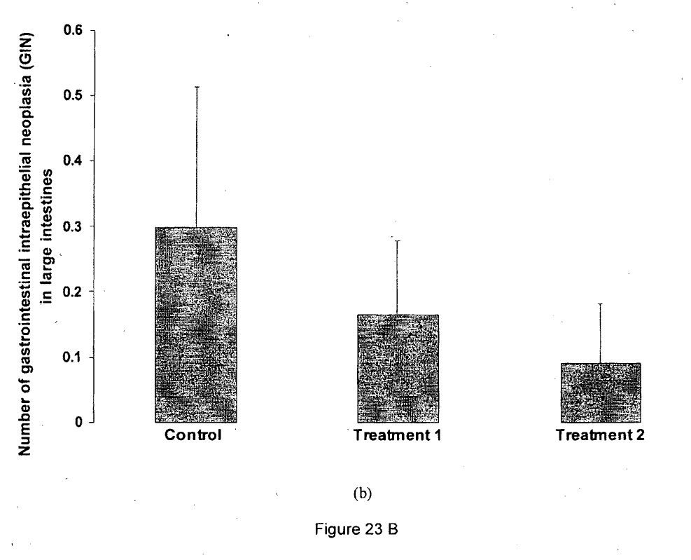

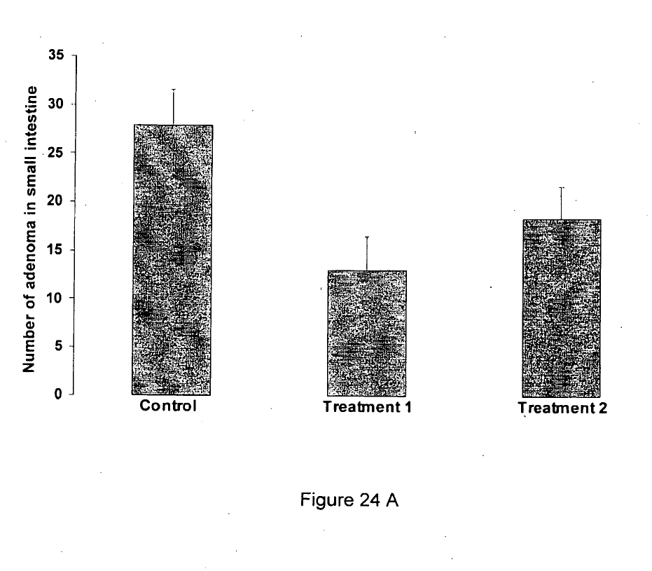

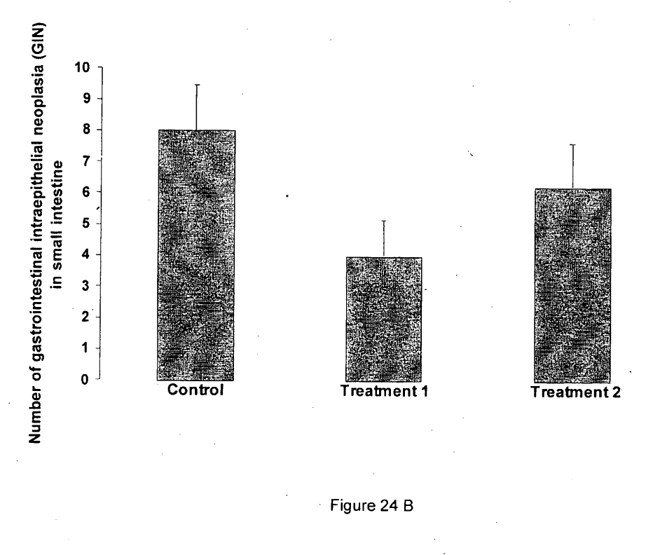

The number of adenomas, both with low and high grade dysplasia and gastrointestinal intraepithelial neoplasias (GIN) were scored for each animal group in small and large intestines. The numbers were averaged per animal in a given group. In the large intestines, there was 0.8 of adenomas found in control group versus 0.4 and 0.7 in treatment 1 and 2 groups, respectively (FIG. 23a). In the small intestines, there were 28 of adenomas found in control group versus 13 and 18 in treatment 1 and 2 groups, respectively (FIG. 24a). In the large intestines, there were 0.3 GIN's found in control group versus 0.2 and 0.1 in treatment 1 and 2 groups, respectively (FIG. 23b). In the small intestines, there were 8 GIN's found in control group versus 4 and 6 in treatment 1 and 2 groups, respectively (FIG. 24b).

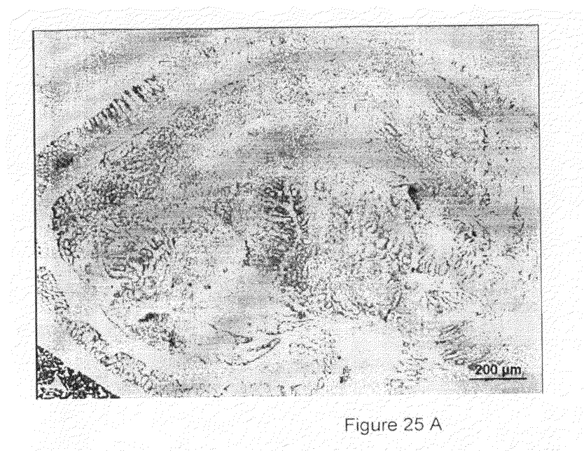

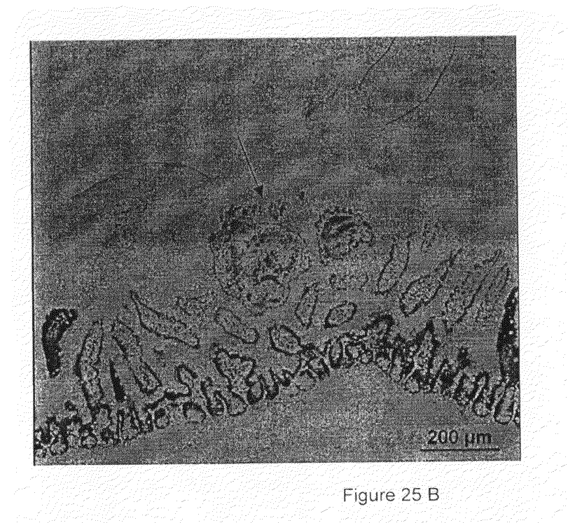

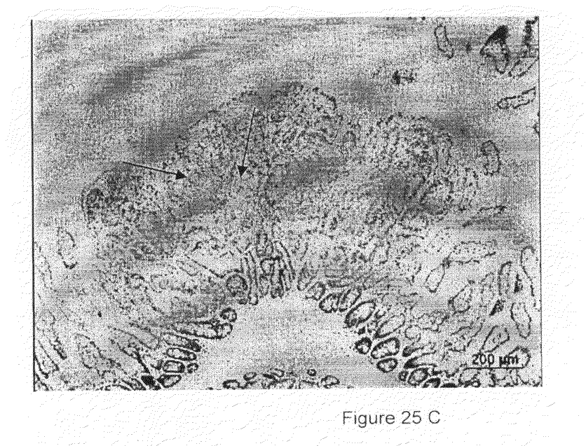

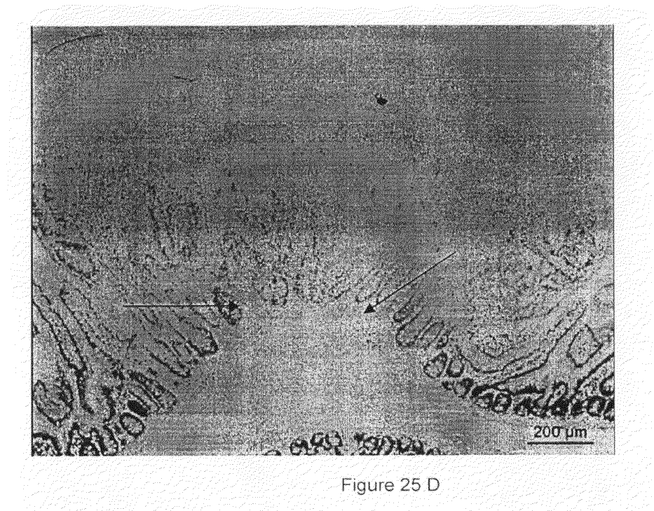

Histological analysis of colon lesions showed that in control the tumors were mostly well differentiated pedunculated adenomas with high grade of dysplasia (FIG. 25a) whereas in mice treated with microencapsulated bacterial probiotic cells the cytological and architectural abnormalities found had either microadenoma, tumors with low grade of dysplasia or broad-based adenoma (FIG. 25 b, c, d).

Analysis

Secretion of IL-6 is strongly associated with the pathogenesis of IBD, and overproduction of IL-6 by intestinal epithelial cells is thought to play a part in the pathogenesis of IBD. IL-6 and TNFa can initiate the innate immune response by inducing the acute phase of inflammation. Additionally, IL-6 also appears to be involved in malignant transformation, tumor progression and tumor-associated cachexia, as reported in studies on Kaposi's sarcoma, multiple myeloma, renal cell carcinoma, prostate cancer, ovarian cancer and breast cancer. Although the difference in the average levels of IL-6 between groups is not statistically significant, an overall decreasing trend was observed for both treatment groups. This indicates that an anti-inflammatory state correlates with the beneficial effect of the probiotic on the involved immunomodulatory mechanisms. We did not observe a beneficial effect of bacterial oral administration at the beginning of treatment.

The presence of blood in feces is one of many symptoms that may indicate the presence of polyps in the colon or rectum, or cancer. The Hemoccult SENSA test was performed at the beginning and the end of treatment. The rectal bleeding was observed in animals on arrival and the test was repeated at the end of experiment to verify whether the treatment has an effect to lower the amount of blood detected. This qualitative test detected the presence of blood in the feces in all animal cages at the end of the treatment which does not reveal any significant changes within the groups.

It is known that bile acids contribute to colonic carcinogenesis by disturbing the fine balance between proliferation, differentiation, and apoptosis in colonic epithelial cells. It is also known that secondary bile acids have been implicated as an important etiological factor in colorectal cancer. Bile acids in the feces act as a promoter of colon cancer, in particular deoxycholic acid (DCA), which is one kind of the secondary bile acid. DCA/cholic acid (CA) ratio in feces is also said to have a diagnostic significance in colon cancer.

It is known that lactobacilli are unable to bind the major conjugated bile acid, glycocholic acid (GCA). Further, it is also known that the colonic microflora probably has bile salt hydrolase (BSH) activity which causes the breakdown of conjugated bile acids to the secondary bile acids, most notably DCA. Results show that L. acidophilus used in this study had no significant BSH activity. Owing to the fact that DCA is the primary bile acid measured in feces using the total bile analysis kit, the overall decreasing trend indicates that a minor amount of primary bile acids are deconjugated to secondary bile acids. This trend could result on account of replacement of a BSH positive colon flora with one that exhibits lesser BSH activity. Our results indicate that microencapsulated bacterial cells can have an influence in tumorigenesis.

The number of adenoma found in each of the treatment groups in the colon and in a small intestine indicates that treatment 1 had a greater impact than treatment 2. However, the number of gastrointestinal intraepithelial neoplasias found in the colon was smaller than in a small intestine. Although there were no statistically significant changes within treatment groups, there was a change between the control and both treatments.

Among all the organs examined in this study there was only one malignant tumor (adenocarcinoma) found in the small intestine of animal from control group. The nuclei were enlarged and pleomorphic with variable loss of their polarity. The glandular structure was distorted and resembled that seen in overt colonic carcinoma. The highest tissue damage was observed in the colon of control group animals under the same conditions as applied to the other tissues. This is probably due to the fact that the colonic wall, including the mucosa and the submucosa, is much thinner than that of the other organs.

Most of the adenoma found were sessile/broad-based and were composed of papillary projections of lamina propria covered by an epithelium. There was no adenoma or lesions found in removed ceca. The greatest loss in mucin secretion was displayed in severely dysplastic glands of control group animals sacrificed at 12^{th} week of the experiment. The glands were closely packed with one another and their structural atypia, e.g., “back to back” arrangement became more prominent. Nuclei were plump but still uniform and smaller than those in carcinomatous glands. Cytological abnormalities detected included cellular and nuclear pleiomorphism and loss of polarity. Architectural abnormalities were the presence of intraglandular papillary projections and of cribriform and solid epithelial areas. However, there were no major differences in animal tissues collected from animals sacrificed at different time periods. The tumors found in both treatment groups showed some features of papillary carcinoma-grooved nuclei and papillary architecture, but these were not consistent.

The present study demonstrates that microencapsulated probiotic bacteria in yogurt formulation exert promising action on polyp progression by delaying the intestinal inflammation and maintaining the constant body weight of Min mice.

In summary, a novel yogurt formulation with microencapsulated acidophilus was devised for targeted delivery of probiotic bacteria for biotherapy in colon cancer.

While the invention has been described in connection with specific embodiments thereof, it will be understood that it is capable of further modifications and this application is intended to cover any variations, uses, or adaptations of the invention following, in general, the principles of the invention and including such departures from the present disclosures as come within known or customary practice within the art to which the invention pertains and as may be applied to the essential features hereinbefore set forth, and as follows in the scope of the appended claims.

1. An oral formulation comprising:
a microcapsule containing bacteria; and
a fermented milk carrier.

2. The oral formulation according to claim 1, wherein said microcapsule comprises a semipermeable capsule comprising poly-L-Lysine (PLL) and alginate and wherein the microcapsule is resistant to degradation in gastrointestinal conditions.

3. The oral formulation according to claim 1, wherein said bacteria are Lactobacilli bacteria or Bifidobacterium bacteria.

4. (canceled)

5. The oral formulation according to claim 1, wherein said bacteria are live.

6. The oral formulation according to claim 1, wherein said bacteria are present in a range from 10⁹ to 10¹² colony forming units per milliliter (CFU/mL).

7. The oral formulation according to claim 1, wherein said microcapsule comprises a material selected from the group consisting of alginate-poly-L-Lysine-alginate (APA), alginate-chitosan (AC), alginate pectinate polylysine pectinate alginate (APPPA), alginate polyethylene glycol alginate (APEGA) and alginate chitosan genipin alginate (ACGA).

8. The oral formulation according to claim 1, wherein said fermented milk carrier comprises a basic pH buffer and protects the bacteria and/or the microcapsule from gastrointestinal fluids.

9. The oral formulation according to claim 8, wherein said basic pH buffer is between pH 6-9.

10. The oral formulation according to claim 1, wherein said fermented milk carrier comprises yogurt.

11. (canceled)

12. (canceled)

13. The oral formulation according to claim 10, wherein the yogurt comprises 1-10 grams of microencapsulated bacteria per 100 grams of yogurt.

14. (canceled)

15. (canceled)

16. (canceled)

17. A method for treatment or prevention of gastrointestinal or urinary tract related disease or disorder, cancer and/or tissue inflammation in a subject in need thereof or for nutritional supplementation of a subject, comprising orally administering to the subject the oral formulation of claim 1.

18. (canceled)

19. (canceled)

20. The method according to claim 17, wherein the subject is a human.

21. (canceled)

22. (canceled)

23. The method according to claim 17, wherein said gastrointestinal disease comprises Inflammatory Bowel Disease (IBD), Crohn's Disease colitis, enteroinvasive colitis, Clostridium difficile colitis, Ulcerative Colitis (UC), Inflammatory Bowel Syndrome (IBS), pouchitis, diverticulitis, gastroenteritis, colic, appendicitis, appendicitis, ascending colangitis, esophagitis, gastritis, or enteritis.

24. (canceled)

25. The method according to claim 24, wherein said cancer comprises breast cancer, colorectal cancer, prostate cancer, lung cancer, colon cancer and inflammation-related colon cancer.

26. (canceled)

27. The method according to claim 17, wherein said tissue inflammation comprises inflammation of tissue in bowel, colon, sigmoid colon, rectum, appendix, anus, esophagus, stomach, mouth, liver, billiary, tract or pancreas.

28. (canceled)

29. (canceled)

30. (canceled)

31. (canceled)

32. (canceled)

33. The method according to claim 17, wherein said urinary tract related disease or disorder comprises a urinary tract infection or a yeast infection.

34. A method of medical treatment of an inflammatory gastrointestinal disease or disorder or an inflammation-related colon cancer in a subject in need thereof, comprising detecting the presence of inflammatory gastrointestinal disease or disorder or inflammation-related cancer in the subject, wherein if inflammatory gastrointestinal disease or disorder or inflammation-related cancer is detected, then administering the formulation of claim 1 to the subject.

35. (canceled)

36. The method of claim 34, comprising detection of: elevated C Reactive Protein (CRP), increased Erythrocyte Sedimentation Rate (ESR), elevated neutrophil count, elevated eosinophil count, elevated monocyte count, elevated white blood cell count (WBC), elevated immunoglobulin count or elevated IgA, compared to a subject not having inflammation.

37. (canceled)

38. (canceled)

39. The method of claim 34, wherein the detecting step comprises determining the presence of cancer in the subject using fecal occult blood (FOB), visible protrusion adenomatous polyps from the mucosal surface, digital rectal exam, colonoscopy, sigmoidiscopy, abdominal series radiograph with contrast, double contrast enema abdominal radiograph or abdominal CT scan.

40. The method of claim 34, wherein the detecting step comprises determining the presence of cancer in the subject with a blood test of the subject comprising detection of elevated carcinoembryonic antigen (CEA) compared to a subject not having cancer.

41. (canceled)

42. (canceled)

43. (canceled)

44. (canceled)
